(19) United States
(12) Patent Application Publication
(10) Pub. No.: US 2025/0026732 A1
(43) Pub. Date: Jan. 23, 2025

(54) AN IMPROVED PROCESS FOR THE PREPARATION OF CRYSTALLINE FORM-A OF CYANTRANILIPROLE

(71) Applicant: NATCO PHARMA LIMITED, Hyderabad (IN)

(72) Inventors: Amarnath JAMPANI, Hyderabad (IN); Veeraiah ARUDRA, Hyderabad (IN); Radha Rani SAMATHAM, Hyderabad (IN); Janaki Rama Rao RAVI, Hyderabad (IN); Durga Prasad KONAKANCHI, Hyderabad (IN); Pulla Reddy MUDDASANI, Hyderabad (IN); Venkaiah Chowdary NANNAPANENI, Hyderabad (IN)

(21) Appl. No.: 18/712,871

(22) PCT Filed: Nov. 22, 2022
(86) PCT No.: PCT/IN2022/051018

§ 371 Date: May 23, 2024

(30) Foreign Application Priority Data
Nov. 24, 2021 (IN) 202141054091

(51) Int. Cl. C07D 401/04

(52) U.S. Cl. CPC C07D 401/04

(57) ABSTRACT

The present invention relates to an improved process for the preparation of Crystalline Form-A of Cyantraniliprole of Formula-I.

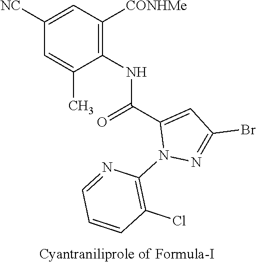

### FIELD OF THE INVENTION

The present invention relates to an improved process for the preparation of Crystalline Form-A of Cyantraniliprole of Formula-I.

### BACKGROUND OF THE INVENTION

3-Bromo-1-(3-chloropyridin-2-yl)-N-(4-cyano-2-methyl-6-(methylcarbamoyl)phenyl)-1H-pyrazole-5-carboxamide in development is highly effective against insects, whose common name is Cyantraniliprole of Formula-I.

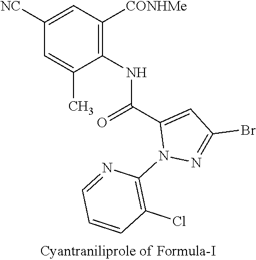

WO 2010056720 A1 discloses that the Crystalline Form-A of Cyantraniliprole can be prepared by dissolving Form-B of Cyantraniliprole in solvent at a temperature between about 40° C. and boiling point of the solvent, wherein solvent is selected from water, n-heptane, 1-chlorobutane, toluene, 1-butanol and 1-pentanol. And Crystalline Form-A of Cyantraniliprole can also be prepared by adding seeding material of Form-A of Cyantraniliprole to Form-B of Cyantraniliprole in solvent.

The above process disclosed in WO 2010056720 A1 suffers from the following disadvantages outlined below:
i) High boiling solvents like 1-buatnol and 1-pentanol were used in some examples, which are difficult to remove from the product.
ii) Seeding material is required to achieve required polymorphic form in some instances.
iii) Required longer hours maintenance time which increased time cycle.
iv) Equipment suitability mentioned in some examples may difficult to adopt on plant scale.

### OBJECTIVE OF THE INVENTION

The main objective of the present invention is to provide a simple and cost effective process for the preparation of Crystalline Form-A of Cyantraniliprole of Formula-I with high purity and good yield on a commercial scale.

### SUMMARY OF THE INVENTION

The present invention relates to an improved process for the preparation of Crystalline Form-A of Cyantraniliprole of Formula-I,

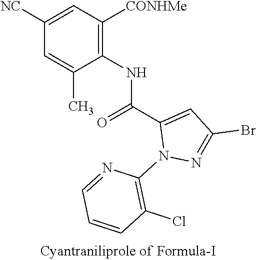

comprising the steps of:
a) suspending crystalline Form-B of Cyantraniliprole in a suitable solvent;
b) heating the reaction mixture to a suitable temperature ranges from 70° C. to 120° C.;
c) cooling the reaction mixture to room temperature;
d) isolating Crystalline Form-A of Cyantraniliprole of Formula-I.

### DETAILED DESCRIPTION OF THE INVENTION

The present invention relates to an improved process for the preparation of Crystalline Form-A of Cyantraniliprole of Formula-I, comprising the steps of:
a) suspending crystalline Form-B of Cyantraniliprole in a suitable solvent;
b) heating the reaction mixture to a suitable temperature ranges from 70° C. to 120° C.;
c) cooling the reaction mixture to room temperature;
d) isolating Crystalline Form-A of Cyantraniliprole of Formula-I.

The suitable solvent used in step-(a) is selected from methyl cyclohexane, xylene or mixture thereof.

In step (b), reaction may be carried out by azeotropic distillation.

In step (b), the reaction may be performed from 50° C. to 125° C. for 1 hours to 12 hours, preferably 80-100° C. for 1 hour to 5 hours.

In step (c), the reaction mixture may be cooled from 25° C. to 30° C. for 1 hours to 12 hours.

in step-(d) isolating refers to the solvent removal by known techniques which are selected from but not limited to removal by distillation, by decanting, by filtration, cooling the clear solution to lower temperatures to precipitate the solid followed by filtration of the reaction.

### BRIEF DESCRIPTION OF THE DRAWINGS

FIG. 1: Illustrates the PXRD pattern of Crystalline Form-A of Cyantraniliprole of Formula-I.

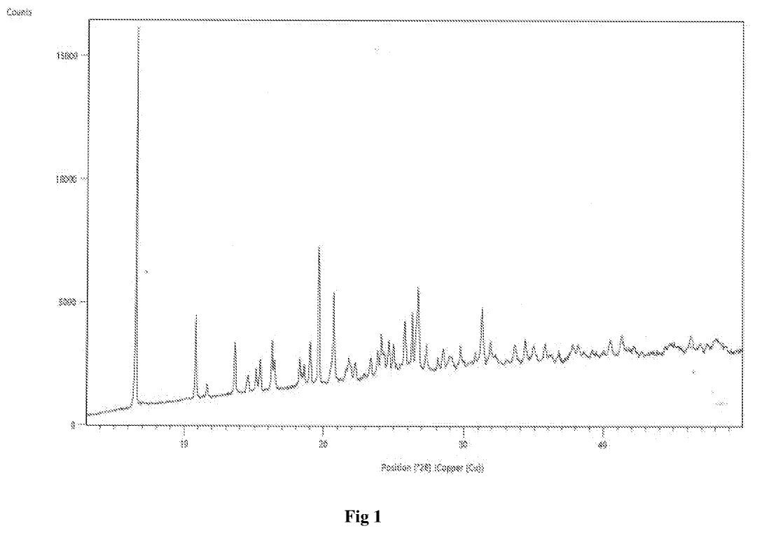

FIG. 2: Illustrates the DSC of Crystalline Form-A of Cyantraniliprole of Formula-I.

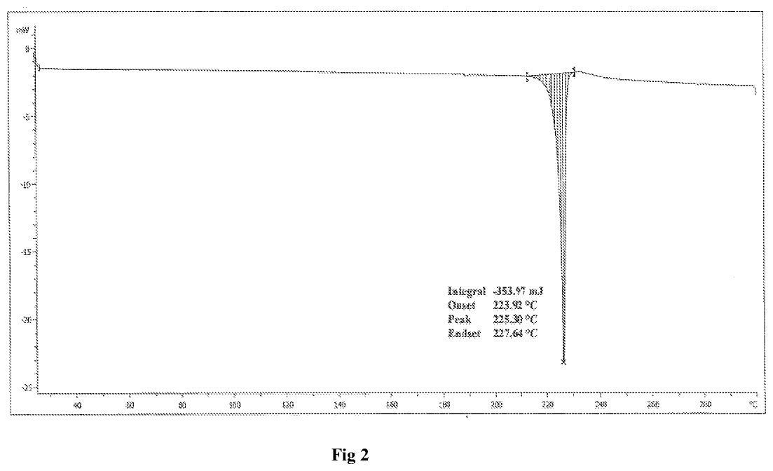

The following examples are provided to illustrate the invention and are merely for illustrative purpose only and should not be construed to limit the scope of the invention.

### Example-1: Preparation of Crystalline Form-A of Cyantraniliprole of Formula-I

Charged Acetonitrile (150.0 ml), 3-bromo-1-(3-chloro-2-pyridinyl)-1H-pyrazole-5-carboxylic acid (50.0 g, 0.165 moles) into 500.0 ml four necked RB flask equipped with condenser, nitrogen bubbler, stopper and thermosocket. Reaction mass was stirred for 5 min at 25 to 35° C. Thionyl chloride (27.5 g, 0.231 moles) was added to the mass slowly through addition funnel during 15 to 30 min by maintaining the mass temperature not crossing 35° C. Reaction mass was heated to 75 to 80° C. and stirred for 3.0 to 3.5 h. After reaction completion, reaction mass was cooled to 25 to 30° C. and kept aside under nitrogen atmosphere (Reaction mass-I).

Charged 2-amino-5-cyano-3,N-dimethylbenzamide (32.8 g, 0.173 moles), acetonitrile (150.0 ml) in to another 1000.0 ml four necked RB flask, equipped with condenser, nitrogen bubbler stopper and thermosocket. Give stirring at 25 to 35° C. Reaction mass was white colour suspension. 3-Picoline (30.73 g, 0.33 moles) was added to the above reaction mass and stirred for 10 to 15 min at 25 to 35° C. Reaction mass was cooled to 0 to 5° C. Reaction mass-I was slowly added to this reaction mass during 1.5 to 2.0 h by maintaining the mass temperature between 0 to 5° C. After completion of addition, reaction mass was stirred for 3.0 to 3.5 h at 0 to 5° C. Then the reaction mass was allowed to 25 to 30° C. and stirred for 8 to 10 h. After completion of maintenance, TLC was checked for reaction completion. After reaction completion, reaction mass was cooled to 0 to 5° C., stirred for 2 h.

Solid was filtered under suction, under nitrogen atmosphere and suction dried for 30 min. The above wet material was leached with acetonitrile (250.0 ml) at 75 to 80° C. for 1 h, then at 25 to 30° C. for 1 h. Solid was filtered under suction under nitrogen atmosphere and suction dried for 30 min.

The resulting solid obtained above was suspended in methyl cyclohexane (1000.0 ml) and heated to reflux, water was removed by Dean-Stark apparatus. After removal of water, the suspension was cooled to 25 to 30° C., stirred for 1.0 h and filtered. Solid was suction dried for 30 min then dried in vacuum oven at 60 to 65° C. for 8 to 10 h. Wt. of the compound: 64.5 g (82.38% by theory)

### Example-2: Preparation of Crystalline Form-A of Cyantraniliprole of Formula-I

Charged Acetonitrile (150.0 ml), 3-bromo-1-(3-chloro-2-pyridinyl)-1H-pyrazole-5-carboxylic acid (50.0 g, 0.165 moles) into 500.0 ml four necked RB flask equipped with condenser, nitrogen bubbler, stopper and thermosocket. Reaction mass was stirred for 5 min at 25 to 35° C. Thionyl chloride (27.5 g, 0.231 moles) was added to the mass slowly through addition funnel during 15 to 30 min by maintaining the mass temperature not crossing 35° C. Reaction mass was heated to 75 to 80° C. and stirred for 3.0 to 3.5 h. After reaction completion, reaction mass was cooled to 25 to 30° C. and kept aside under nitrogen atmosphere (Reaction mass-I).

Charged 2-amino-5-cyano-3,N-dimethylbenzamide (32.8 g, 0.173 moles), acetonitrile (150.0 ml) in to another 1000.0 ml four necked RB flask, equipped with condenser, nitrogen bubbler stopper and thermosocket. Give stirring at 25 to 35° C. Reaction mass was white colour suspension. 3-Picoline (30.73 g, 0.33 moles) was added to the above reaction mass and stirred for 10 to 15 min at 25 to 35° C. Reaction mass was cooled to 0 to 5° C. Reaction mass-I was slowly added to this reaction mass during 1.5 to 2.0 h by maintaining the mass temperature between 0 to 5° C. After completion of addition, reaction mass was stirred for 3.0 to 3.5 h at 0 to 5° C. Then the reaction mass was allowed to 25 to 30° C. and stirred for 8 to 10 h. After completion of maintenance, TLC was checked for reaction completion. After reaction completion, reaction mass was cooled to 0 to 5° C., stirred for 2 h.

Solid was filtered under suction, under nitrogen atmosphere and suction dried for 30 min. The above wet material was leached with acetonitrile (250.0 ml) at 75 to 80° C. for 1 h, then at 25 to 30° C. for 1 h. Solid was filtered under suction under nitrogen atmosphere and suction dried for 30 min.

The resulting solid obtained above was suspended in xylene (1000.0 ml) and heated to reflux, water was removed by Dean-Stark apparatus. After removal of water, the suspension was cooled to 25 to 30° C., stirred for 1.0 h and filtered. Solid was suction dried for 30 min then dried in vacuum oven at 60 to 65° C. for 8 to 10 h. Wt. of the compound: 63.0 g (80.46% by theory)

We claim:

1. An improved process for the preparation of Crystalline Form-A of Cyantraniliprole of Formula-I, comprising the steps of: 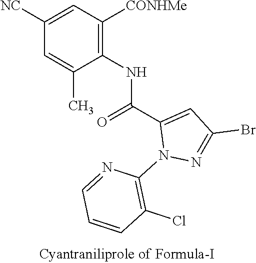
a) suspending crystalline Form-B of Cyantraniliprole in a suitable solvent;
b) heating the reaction mixture to a suitable temperature ranges from 70° C. to 120° C.;
c) cooling the reaction mixture to room temperature;
d) isolating Crystalline Form-A of Cyantraniliprole of Formula-I.

2. The process as claimed in claim 1, wherein the suitable solvent used in step-(a) is selected from methyl cyclohexane and xylene or mixture thereof.

3. The process as claimed in claim 1, wherein the step-(b) is carried out by azeotropic distillation.

4. The process as claimed in claim 1, wherein isolating Crystalline Form-A of Cyantraniliprole of Formula-I is carried out by conventional techniques such as filtration and dried the product.
